(19) United States
(12) Patent Application Publication
(10) Pub. No.: US 2009/0062774 A1
(43) Pub. Date: Mar. 5, 2009

(54) Device For Introducing A Catheter By The Umbilical Route, And Kit

(75) Inventors: Dominique Haumont, Bruxelles (BE); Fabien Letang, Bethisy-Saint-Pierre (FR)

Correspondence Address: LEVINE & MANDELBAUM, 222 Bloomingdale Road, Suite 203, WHITE PLAINS, NY, 10605, US

(73) Assignee: VYGON, Ecouen (FR)

(21) Appl. No.: 12/281,907

(22) PCT Filed: Mar. 15, 2007
(86) PCT No.: PCT/FR07/00445

§ 371 Date: Sep. 5, 2008

(30) Foreign Application Priority Data
Mar. 16, 2006 (FR) 0602300

(51) Int. Cl. A61M 25/088

(52) U.S. Cl. 604/528

(57) ABSTRACT

The invention relates to a device for introducing a silicone catheter by the umbilical route. The introducing device comprises a polyurethane catheter in which the silicone catheter can slide, and a rectilinear and rigid tube (4) which is made of stainless metal and is shorter than the catheter (6) and whose internal diameter is adapted to the external diameter of the silicone catheter (1) in order to allow the silicone catheter to slide in the metal tube, while preventing entry of air between them, this metal tube being introduced into the proximal end of the polyurethane catheter and fixed to said catheter in such a way as to prevent entry of air between them. The invention applies in particular to the catheterization of premature babies or neonates.

The invention relates to a device for introducing a central venous catheter by the umbilical route in premature babies and neonates.

For this application, it has been proposed to use a silicone catheter introduced by means of a polyurethane umbilical catheter (Amata M, Weissmüller A, Huppi P: “Umbilicous venous silastic catheter—a new procedure in low-birth-weight infants” Neonatologica 1992 (2), 117-20, and Scharrer B, Rudin C, Nars P W: “Transumbilical venous access with small diameter silastic catheters in very low birth weight infants” Eur J Pediatr. 1997, 156(11): 897-8).

Silicone catheters are tolerated better by bodies than those made of polyurethane. On the other hand, they are very soft and, for this reason, difficult to introduce. Via the umbilical route, the major veins of neonates, which are subject to negative pressure, are touched, and the risk of air embolism due to entry of air is high particularly in cases of deep inspiration. Air embolism is fatal.

Polyurethane catheters are relatively less flexible than silicone catheters but it is difficult to ensure the progression of a silicone catheter in a polyurethane catheter if both catheters are sufficiently adapted with respect to each other to prevent entry of air between the catheters.

The present invention relates to a device for introducing a silicone central venous catheter by the umbilical route, in premature babies or neonates, comprising a polyurethane catheter and facilitating the sliding of the silicone catheter in the polyurethane catheter while preventing the risks of air embolism.

According to the invention, the introduction device, which comprises a polyurethane catheter in which the silicone catheter can slide, also comprises a section of rectilinear and rigid tube, which is made of stainless metal and is shorter than the catheter and whose internal diameter is adapted to the external diameter of the silicone catheter in order to allow the silicone catheter to slide in the metal tube, while preventing entry of air between them, this section of metal tube being introduced into the proximal end of the polyurethane catheter and fixed to said catheter in such a way as to prevent entry of air between them.

The stainless metal tube is ideal, as it enables the necessary precision (+/−0.01 mm) and offers satisfactory sliding in contact with the silicone catheter.

Due to its rigidity, the metal tube makes it possible to introduce the silicone catheter without said catheter being blocked. It forms an external mandrel. It is possible to push on the silicon catheter which can only remain straight. The silicone catheter simply needs to travel a length in the polyurethane catheter of approximately 3 to 6 cm before reaching the vein and being “aspirated” by the slight negative pressure (return of blood to heart).

For the introduction of a silicone catheter having an internal diameter of 0.3 mm and external diameter of 0.6 mm, 15 to 50 cm long, the introduction device according to the invention consists of:
a stainless metal tube 0.8×1.0 mm in diameter and approximately 45 mm in length,
a 5 Fr polyurethane catheter 1.0×1.7 mm in diameter and 5 to 10 cm in length.

Preferentially, the metal tube is equipped with a flexible base with two fins to grip the introduction device. It is introduced by clamping and bonding in the polyurethane catheter (which also displays several X-ray detectable opaque radio lines). The polyurethane catheter is sufficiently long to enter at the desired point in the umbilical vein. According to the size of the babies, two lengths are provided: 6 to 9 cm.

The distal end of the polyurethane catheter may be blunted if required to avoid any aggressive shape, and said catheter may be marked in centimetres to indicate the length inserted into the vein.

The metal tube enables pushing and the non-entry of air due to the rigidity, sliding and precise tolerances thereof.

The introduction device is used to push the very thin and very soft silicone catheter without any risk of being stuck, it makes it possible to push the silicone catheter and remove the introduction device without any risk of air embolism.

In the figures of the drawing attached:

FIG. 1 is a schematic plane view of an introduction device according to the invention;

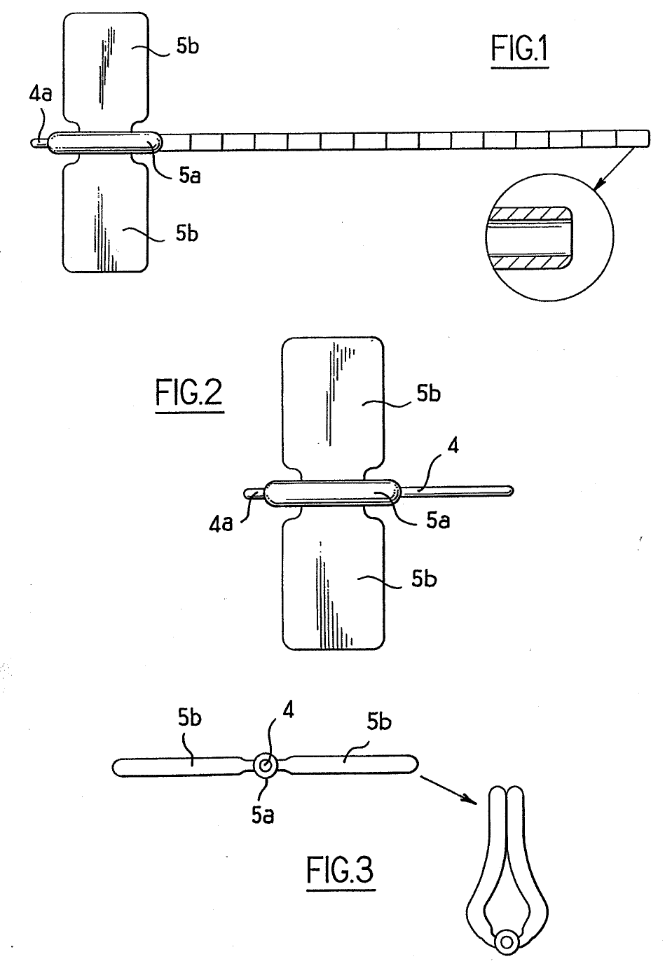

FIG. 2 is a plane diagram of the metal tube;

FIG. 3 is a cross-section of the metal tube on the gripping fins;

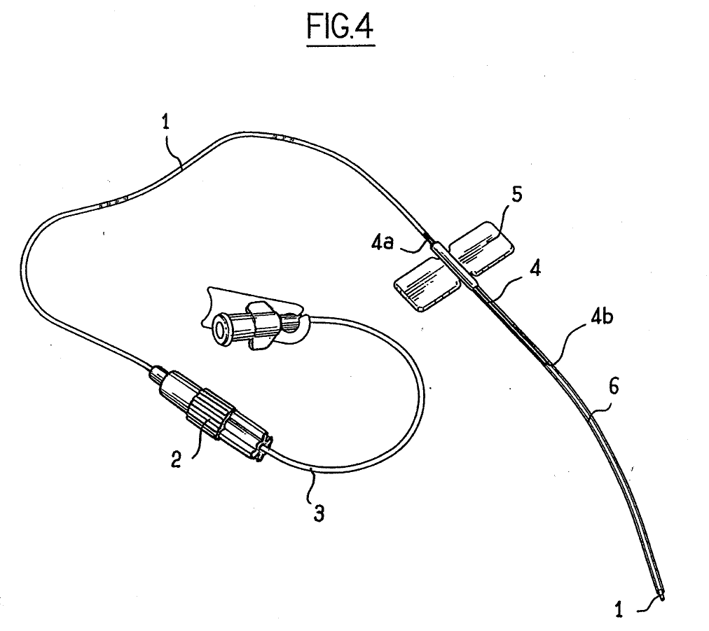

FIG. 4 is a view of a kit consisting of the introduction device in FIG. 1 and a silicone catheter, and

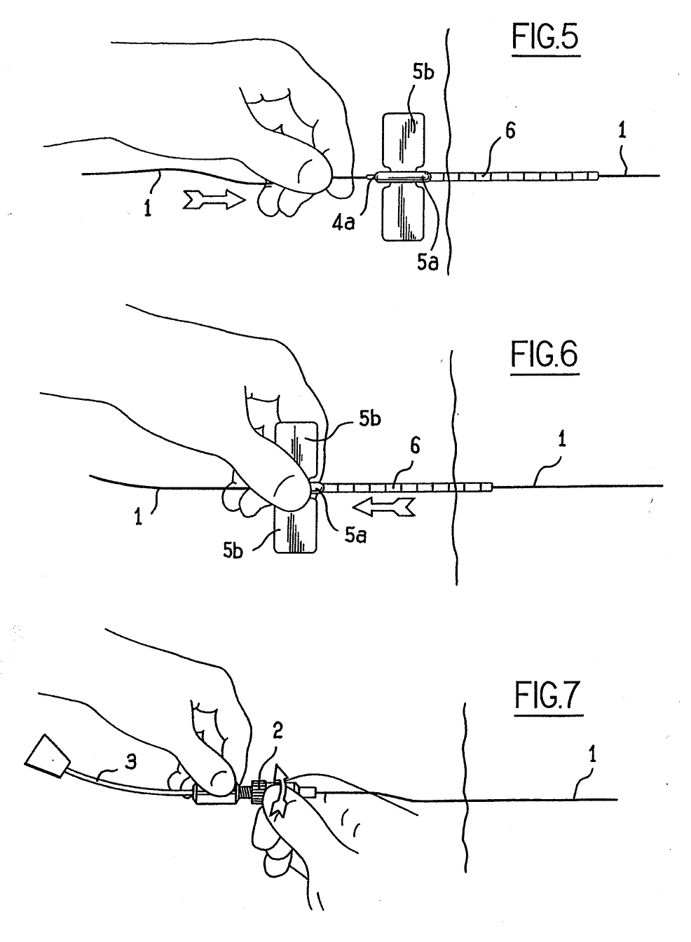

FIGS. 5, 6 and 7 are diagrams of use.

The introduction device comprises a relatively short stainless metal rectilinear tube 4 comprising a proximal end 4a equipped with a gripping base 5 consisting of a shaft 5a and two fins 5b.

The metal tube is introduced into one end of a flexible polyurethane catheter 6 which is substantially longer than the metal tube. The tube and the catheter are fixed by bonding.

For the positioning thereof, the silicone catheter is inserted into the metal tube and then into the polyurethane catheter (FIG. 5). In order to remove the metal tube and the polyurethane tube, this assembly is held by the fins and slid to the rear onto the silicone catheter (FIG. 6).

The silicone catheter 1 after the removal of the polyurethane catheter may be equipped with a removable proximal base 2 enabling the connection thereof to a device 3 (FIG. 7).

The invention is not limited to this example of an embodiment. In particular, the materials cited could be replaced by functionally equivalent materials or materials having superior qualities.

The invention is not limited to the embodiment described.

1. Device for introducing a silicone catheter, by the umbilical route, which comprises a flexible polyurethane catheter in which the silicone catheter can slide, characterised in that it also comprises a section of rectilinear and rigid tube, which is made of stainless metal and is shorter than the catheter and whose internal diameter is adapted to the external diameter of the silicone catheter in order to allow the silicone catheter to slide in the metal tube, while preventing entry of air between them, this metal tube being introduced into the proximal end of the polyurethane catheter and fixed to said catheter in such a way as to prevent entry of air between them.

2. Introduction device according to claim 1 wherein the stainless metal tube is provided with a proximal base with fins.

3. Introduction device according to claim 1 wherein the polyurethane catheter displays X-ray detectable opaque radio lines.

4. Kit comprising an introduction device according to claim 1 and a silicone catheter, the internal diameter of the stainless metal tube being adapted to the external diameter of the silicone catheter to enable the sliding of the catheter while preventing entry of air between them.

5. Kit according to claim 4 wherein the silicone catheter is equipped with a removable proximal base.

6. Kit according to claim 4 comprising:
a 2 Fr silicone catheter (0.3×0.6 mm) 15 to 50 cm in length,
a stainless metal tube 0.8×1.0 mm in diameter and approximately 45 mm in length,
a 5 Fr polyurethane catheter 1.0×1.7 mm in diameter and 5 to 10 cm in length.
